(19) United States
(12) Patent Application Publication
(10) Pub. No.: US 2007/0275104 A1
(43) Pub. Date: Nov. 29, 2007

(54) Food Compositions and Methods of Treating Periodontal Disease

(76) Inventors: Kenneth S. Kornman, Newton, MA (US); Leon Wilkins, North Andover, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/748,129

(22) Filed: May 14, 2007

Related U.S. Application Data
(60) Provisional application No. 60/799,851, filed on May 12, 2006.

(51) Int. Cl. A61K 36/82; A61K 36/73; A61K 36/53

(52) U.S. Cl. 424/729; 424/745; 424/765; 424/769

(57) ABSTRACT

The invention provides a method of alleviating a sign or symptom of periodontal disease in a subject, by administering to the subject a composition containing a natural compound which inhibits matrix metalloproteinase activity and interleukin-1 activity.

### RELATED APPLICATIONS

This application claims the benefit of U.S. Ser. No. 60/799,851 filed May 12, 2006 the contents of which is incorporated herein by reference in its entirety.

### FIELD OF THE INVENTION

The invention relates to the use of food compositions for the treatment of periodontal disease.

### BACKGROUND OF THE INVENTION

Periodontal disease (gum disease) is a prevalent health problem in adult humans and animals which, if not controlled or treated, can result in tooth loss. Gingivitis is the more common form of the various gum diseases caused by bacteria on the teeth and gums. When the bacteria are not removed on a daily basis, the bacteria accumulate to form “dental plaque,” a thick mat of bacteria adhering to the tooth surface. In the early stages, gingivitis can cause minor symptoms such as bad breath and bleeding gums.

If not controlled, the bacterial populations that cause gingivitis change to a more virulent dental plaque that extends below the gingiva inducing more extensive inflammation of the gums with loss of bone and connective issues that support the teeth. This form of gum disease in which destruction of bone and connective tissues occurs is called “periodontitis.”

Regular flossing and brushing of the teeth are generally recommended as a preventative for gum disease. Brushing and flossing of the teeth removes bacterial plaque on the surfaces of the teeth and, when carried out on a regular basis, can prevent or reduce periodontal disease. However, when the plaque matures undisturbed and extends subgingivally, it often calcifies forming calculus deposits which are difficult to remove by brushing, flossing, or other patient-applied oral hygiene measures. Typically, these calcified dental plaques must then be removed by a dentist or dental hygienist, by a process called tooth scaling.

Most individuals, even with less than ideal plaque control, develop only gingivitis or mild periodontitis. Some individuals develop a generalized moderate to severe periodontitis which eventually can lead to loss of teeth with compromised mastication and phonetic function. Many individuals with generalized moderate to severe periodontitis have systemic risk factors that increase their susceptibility to periodontal disease. These risk factors include smoking, diabetes mellitus, obesity, osteopenia and low dietary calcium, stress, and genetic factors. National surveys carried out in the last decade show that 8-13% of the U.S. population has severe generalized periodontitis, and approximately 35% have mild to moderate periodontitis.

The main mechanism of tissue destruction in periodontal disease is excess inflammation triggered by dental plaque, although some of the bacteria also can cause direct tissue destruction. The main tissue-destroying inflammatory factors include proinflammatory cytokines, such as IL1, TNF-α, and IL6, as well as tissue-destroying enzymes such as the matrix metalloproteinases.

Various compositions in the form of toothpaste and oral rinses have been proposed to reduce bacterial plaque on the surfaces of the teeth. Current treatment is focused on attacking the harmful bacteria, which are now well recognized as the main contributors to periodontal disease. Treatment protocols presently include thorough scaling of the teeth, with planing of the roots to remove adherent plaque and calculus. This is often augmented with antibiotic powder and gels applied by syringe into the infected gum space, chemical rinses, time-released antimicrobial chips inserted into the infected gum space, chemical flushes of the infected gum space, and stronger oral antibiotics. These invasive chemical and antibiotic approaches augment the resolution of infection, inflammation and healing seen with scaling and root planing. In extensive cases of periodontitis, scaling and root planing may not be adequate and surgical procedures may be necessary to resolve the infection and restore lost tissues. Though the antibiotic approaches do help the periodontal tissues, they also have inherent problems and undesirable side effects including development of antibiotic resistant bacteria. There is a continuing need in the health care field for an improved and effective composition for treating periodontal disease.

### SUMMARY OF THE INVENTION

The invention features broad-spectrum inhibitor of metalloproteases of the collagenase or gelatinase type, combined with inhibitors of IL-1 cytokine production and/or activity, for the treatment of humans or of mammals suffering from a condition or a disease linked to excess or pathological degradation of collagen or of another extracellular support macroprotein, or any other diseases linked to excessive expression of these proteolytic enzymes.

More specifically, the invention features compositions and methods of preventing or alleviating a sign or symptom of periodontal disease. Periodontal disease is prevented or treated by identifying a subject suffering from or at risk of developing periodontal disease and administering to the subject a composition containing a natural matrix metalloproteinase (MMP) inhibitor and a natural interleukin-1 inhibitor. The subject is a mammal such as human, or a non-human primate. The subject is suffering from or at risk of developing periodontal disease. A subject suffering from or at risk of developing periodontal disease is identified by methods known in the art.

The invention provides a food composition containing a natural matrix metalloproteinase (MMP) inhibitor and a natural interleukin-1 inhibitor. A natural MMP inhibitor includes for example Pomegranate Extract, Green Tea Extract, Rosemary Extract, Quercetin, Amla Extract or Kakadu Concentrate. A natural interleukin-1 inhibitor includes for example rose hips or boswellia. The composition is a liquid, a lozenge, a tablet, a chew, a powder or a bar.

Optionally, the minimum matrix metalloproteinase inhibitory concentration of the natural ingredient in the presence of rosehips is synergistically less than the minimum matrix metalloproteinase inhibitory of the natural extract alone. Alternatively, the minimum interleukin-1 inhibitory concentration of the rose hips in the presence of the natural ingredient is synergistically less than the minimum interleukin-1 inhibitory of the rose hips alone.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### BRIEF DESCRIPTION OF THE DRAWINGS

FIG. 1 are tables summarizing MMP inhibition results.

FIG. 2 are tables summarizing the results of the interference studies.

FIG. 3 are tables summarizing the results of the interference studies.

FIG. 4 is a line graph showing extract inhibition of MMP-2.

FIG. 5 is a line graph showing extract inhibition of MMP-9.

FIG. 6 is a line graph showing extract inhibition of MMP-8.

FIG. 7 is a line graph showing inhibitor interaction of MMP-2.

FIG. 8 is a line graph showing inhibitor interaction of MMP-9.

FIG. 9 is a line graph showing inhibitor interaction of MMP-8.

### DETAILED DESCRIPTION

The invention is based in part on the discovery that natural matrix metalloprotease inhibitors in combination with a natural interleukin-1 (IL1) inhibitor can achieve a preventative or therapeutic effect on periodontal disease. Accordingly, the invention provides compositions and methods for preventing, treating or alleviating a sign or symptom of periodontal disease. Specifically, the anti-periodontal disease compositions include a natural matrix metalloprotease inhibitor. The natural metalloprotease inhibitor is a plant extract or a mixture of extracts such as Pomegranate Extract, Green Tea Extract, Rosemary Extract, Quercetin, Amla Extract or Kakadu Concentrate. The natural IL1 inhibitor is for example rosehips, boswellia or other natural IL1 inhibitors known in the art. Periodontal diseases, including gingivitis and periodontitis, are serious infections that, left untreated, can lead to tooth loss. Periodontal disease can affect one tooth or many teeth. It begins when the bacteria in plaque causes the gums to become inflamed.

In the mildest form of the disease, gingivitis, the gums redden, swell and bleed easily. There is usually little or no discomfort. Gingivitis is often caused by inadequate oral hygiene. Untreated gingivitis can advance to periodontitis. With time, plaque can spread and grow below the gum line. Toxins produced by the bacteria in plaque irritate the gums. The toxins stimulate a chronic inflammatory response in which the body in essence turns on itself and the tissues and bone that support the teeth are broken down and destroyed. Gums separate from the teeth, forming pockets (spaces between the teeth and gums) that become infected. As the disease progresses, the pockets deepen and more gum tissue and bone are destroyed. Eventually, teeth can become loose and may have to be removed.

The main cause of periodontal disease is bacterial plaque. However, factors such as tobacco use, genetics, pregnancy, puberty, stress, medications, diabetes, and diet affect the health of your gums and increase your risk of developing periodontal disease.

There are many forms of periodontal disease. The most common ones include gingivitis, aggressive periodontitis, chronic periodontitis, periodontitis as a manifestation of a systemic disease and necrotizing periodontal disease.

Gingivitis is the mildest form of periodontal disease. It causes the gums to become red, swollen, and bleed easily. There is usually little or no discomfort at this stage.

Aggressive Periodontitis is a form of periodontitis that occurs in patients who are otherwise clinically healthy. Common features include rapid attachment loss and bone destruction.

Chronic Periodontitis is a form of periodontal disease resulting in inflammation within the supporting tissues of the teeth, progressive attachment and bone loss and is characterized by pocket formation and/or recession of the gingiva. It is recognized as the most frequently occurring form of periodontitis. It is prevalent in adults, but can occur at any age. Progression of attachment loss usually occurs slowly, but periods of rapid progression can occur.

Periodontitis, often with onset at a young age, associated with one of several systemic diseases, such as diabetes.

Necrotizing Periodontal Disease is an infection characterized by necrosis of gingival tissues, periodontal ligament and alveolar bone. These lesions are most commonly observed in individuals with systemic conditions including, but not limited to, HIV infection, malnutrition and immunosuppression.

Periodontal disease is diagnosed by known methods. For example, sulcus depths are measured around each tooth. There are six measurements taken around the circumference of each tooth with a calibrated straight instrument. Measurements of 4 mm and over are considered unhealthy and diseased. Additionally, a bleeding analysis may be done and rated on a scale of 0-5, based on the amount of bleeding found during measuring of the sulcus depth Zero [“0”] being no bleeding, 3 being moderate bleeding, 5 being severe and uncontrolled bleeding. Bleeding is indicative of some stage of periodontal involvement. Optionally, a breath analysis is done based on the patient's perception of their oral odor. The analysis is rated on a scale 0-5. Zero [“0”] is no odor, 3 is moderate odor, 5 is severe odor. Breath odor is a result of a harmful bacteria buildup in the sulcus and on the tongue. Breath odor is indicative of some stage of periodontal involvement.

A nutritional composition is a composition that comprises naturally occurring components, preferably found in the food supply, that can be sold over the counter, as supplements, functional foods or food ingredients i.e., without a physician's or veterinarian's prescription. A nutritional composition may also be a medical food, intended for the dietary management of a disease or condition for subjects under the supervision of a physician or veterinarian. A pharmaceutical composition is one that includes ethical pharmaceuticals and which requires a physician's or veterinarian's prescription for administration.

A synergist is defined as an agent or compound which when present results in a greater-than-additive increase, augmentation or enhancement of the effect of an agent or compound. In some cases, it may be difficult to determine which compound in a mixture is of primary importance and which only secondary. Thus, in a synergistic mixture of compounds, any of the active compounds within the mixture can be considered a synergist. A composition comprising “synergistic activity” or a “synergistic mixture” is a combination of compounds wherein the combined effect is greater than additive of the individual effects. Synergism may be apparent only at some ranges or concentrations.

By amounts effective to reduce gum damage associated with periodontal disease is meant that the matrix metalloproteinase inhibitor and the interleukin-1 inhibitor is present in a final concentration sufficient for reducing signs or symptoms associated with periodontal disease. This amount includes, but is not limited to, a concentration which acts as a complete prophylaxis or treatment for a symptom of periodontal disease. An “effective amount” is an amount sufficient to effect beneficial or desired results. An effective amount can be administered in one or more administrations. For purposes of this invention, an effective amount is an amount that is sufficient to ameliorate, stabilize, reverse, slow or delay the progression of injury(ies) in subjects i) at risk for a periodontal disease, or ii) associated with, or due to periodontal disease.

An effective amount is an amount less than a standard dose. Alternatively, an effective amount of a therapeutic compound is an amount greater than a standard dose. A standard dose is the amount typically administered to a subject to treat (i.e., alleviate a sign or symptom) the therapeutics' common indication. (i.e., a non-periodontal disease indication). Effective doses vary, as recognized by those skilled in the art, depending on route of administration, excipient usage, and coadministration with other therapeutic treatments including use of other anti-inflammatory agents, or therapeutic agents for treating, preventing or alleviating a sign or symptom of a periodontal disease. A therapeutic regimen is carried out by identifying a mammal, e.g., a human patient suffering from (or at risk of developing) periodontal disease, using standard methods.

A matrix metalloproteinase inhibitor is a compound that decreases matrix metalloproteinase expression or activity. Metalloproteinase inhibitors are known in the art or are identified using methods described herein. A decrease in metalloproteinase expression or activity is defined by a reduction of extracellular matrix turnover (i.e., degradation and remodeling) or a reduction in the expression of MMP or MMP mRNA levels. Matrix metalloproteinase activity is measured for example by using a readily available commercial kit such as the MT-MMP Activity Assay Kit (CHEMICON), or MMP Quantizyme™ Assay System (BIOMOL). Matrix metalloproteinase expression is determined for example by measuring the level pf the polypeptide using e.g., immunoassays based on antibodies to MPP proteins. Alternatively, matrix metalloproteinase expression is determined by measuring the level of MPP mRNAs in, e.g., northern blot hybridization analyses or MPP nucleic acids in, e.g., amplification-based detection methods such as reverse-transcription based polymerase chain reaction. Exemplary matrix metalloproteinase inhibitors include for example, Doxycycline, Pomegranate Extract, Green Tea Extract, Rosemary Extract, Quercetin, Amla Extract or Kakadu Concentrate.

Pomegranate Extract

Extracts from the constituents of pomegranate, i.e., seeds or the inner or outer peel, may be made by methods commonly known in the art. For example, the seeds or the inner or outer peel of pomegranate may be diluted in water and the extract may be made by crushing, squeezing, or extensive vortexing. The insoluble materials of the extract may be separated from the soluble supernatant of the extract. Preferably, the supernatant of the extract is used for the purpose of the present invention, although any oily, lipidic fraction of the extract may also be used. The extract from constituents of pomegranate may be concentrated or diluted, or mixed with each other or with pomegranate juice extract. The extract of pomegranate of the present invention may be in a liquid or solid form. In accordance with one embodiment of the present invention, a solid form of the extract may be made by lyophilizing the liquid extract of the present invention. Alternatively, the constituents of the pomegranate, such as seeds, inner or outer peels, or any insoluble portion discussed above, may be processed directly to form the solid form of the extract of the present invention.

Dried pomegranate seeds contain the steroidal estrogen estrone, the isoflavonic phytoestrogens genistein and daidzein and the phytoestrogenic coumestrol. In pomegranate juice, fructose and glucose are present in similar quantities, calcium is 50% of its ash content and the principal amino acids are glutamic and aspartic acid. Content of soluble polyphenols in pomegranate juice varied within the limits of 0.2% to 1.0%, depending on variety, and include mainly anthocyanins (such as cyanidin-3-glycoside, cyanidin-3,3-diglycoside and delphindin-3-glucosid), catechins, ellagic tannins, and gallic and ellagic acids.

Green Tea Extract

Green tea extracts are useful in the compositions of the present invention. In some embodiments of the compositions of the invention, the Green tea extract is standardized for polyphenols. For example, Green tea, 98% polyphenols containing 45% polyphenols such as polyphenol (−)-epigallocatechin gallate (EGCG) is prepared from the leaf of the tea herb Camellia sinensis. Polyphenols, e.g., EGCG, in green tea are useful to protective against certain cancers, and they are also potent antioxidants. Green tea preparations are useful to promote immune function and to prevent and treat high cholesterol, heart disease, infection (e.g., Staphylococcus aureus infection, skin infection, bacterial infection, viral infection), acne, aging, immune disorders, dental caries, periodontitis, halitosis, dandruff, cancer, cardiovascular disease (e.g., hypertension, thrombosis, arteriosclerosis), diabetes, elevated blood glucose, diseases of the alimentary canal and respiratory system, influenza hepatitis, liver disease. Green tea extracts are commercially available, e.g., Hunan Kinglong Bio-Resource Co., Ltd., (Xingsha, Changsha, Hunan, P. R. China).

Rosemary Extract

Rosemary leaf contains phenolic acids (2-3% rosemarinic, chlorogenic, and caffeic), phenolic di- and tri-terpenoids (up to 4.6% carnosol, rosmaridiphenol, rosmanol), flavonoids, and essential oils. Carnosic acid, a potent antioxidant, has the unique capability of progressing through several stages of oxidation while continuing to quench free radicals (the “carnosic acid cascade”). Preferably, the rosemary extract contains at least 6%, 10%, 20%, 30%, 35%, 40%, 50%, 60% or more carnosic acid.

Quercetin

Quercetin is a flavonoid that forms the “backbone” for many other flavonoids, including the citrus flavonoids rutin, hesperidin, naringin and tangeritin. Quercetin is found to be the most active of the flavonoids in studies, and many medicinal plants owe much of their activity to their high quercetin content.

Amla Extract

The amla fruit is an Indian medicinal plant. The tincture from amla fruit mainly contains, in high concentrations, tannins, mucic acid, various fruit sugars and a number of free amino acids as well as naturally stabilized vitamin C. It has been shown to be protective against induced liver damage in rat models.

Kakadu Concentrate

The kakadu plum has a high ascorbic acid content. Moreover, the kakadu plum fruit includes appreciable amounts of phytochemicals, for example, gallic acid, ellagic acid, and related compounds. Accordingly, in addition to providing enhanced ascorbic acid levels, the kakadu plum fruit provides many other beneficial phytochemicals.

Natural Food Supplements

The present invention provides a natural food supplement, e.g., a nutraceutical made from plant extracts. The food supplement contains a matrix metalloproteinase and interleukin-1 inhibitory activity that is greater than the matrix metalloproteinase and interleukin-1 inhibitory activity found in the natural plant. The plant extract can be presented in a powdered, liquid, or solid form.

A “nutraceutical” is any functional food that provides an additional benefit other than its nutritional benefit. This category may include nutritional drinks, diet drinks (e.g., SlimfaSt™, BooSt™ and the like) as well as sports herbal and other fortified beverages. The present invention provides nutraceutical compositions that may be used as an anti-periodontal disease agent.

The natural food supplement is likely a reconstitutable powder composition that, when reconstituted with, for example, water, milk or some other similar liquid will provide a drink, which may be used to provide matrix metalloproteinase and interleukin-1 inhibitory activity to a subject in need thereof The powdered composition and drink prepared therefrom are especially useful as an enterally administered component in a program of periodontal disease management which utilizes a number of carefully designed products in various forms, i.e., in shake, soup, fruit drink, snack bar and other solid forms such as tablets, gel caps, and the like, which can be mixed and matched over a period of periodontal disease to provide more attractive and, therefore, more effective support to a patient, particularly those in extended care situations.

In addition to drinks, the natural food supplement of the present invention may be used in foodstuffs. Such plant extracts may be combined with any other foodstuff, for example, oils containing the extracts of this invention may be used as cooking oil, frying oil, or salad oil and may be used in any oil-based food, such as margarine, mayonnaise or peanut butter. Grain flour fortified with the compounds of this invention may be used in foodstuffs, such as baked goods, cereals, pastas and soups. Advantageously, such foodstuffs may be included in low fat, low cholesterol or otherwise restricted dietary regimens.

In addition to the purified plant extract, the nutraceutical or foodstuff also may contain a variety of other beneficial components including but not limited to essential fatty acids, vitamins and minerals. Optional additives of the present composition include, without limitation, pharmaceutical excipients such as magnesium stearate, talc, starch, sugars, fats, antioxidants, amino acids, proteins, nucleic acids, electrolytes, vitamins, derivatives thereof or combinations thereof.

These components should be well known to those of skill in the art, however, without being bound to any particularly formulations or content the present section provides a brief discussion of components that could form part of the food supplements of the present invention. Additional disclosure describing the contents and production of nutritional supplements may be found in e.g., U.S. Pat. No. 5,902,797; U.S. Pat. No. 5,834,048; U.S. Pat. No. 5,817,350; U.S. Pat. No. 5,792,461; U.S. Pat. No. 5,707,657 and U.S. Pat. No. 5,656,312 (each incorporated herein by reference.)

In addition, vitamin C, vitamin B1 (thiamin), and vitamin E also can be provided. Vitamin C requirements are increased in smokers and cigarette smoking is a major contributor to periodontal disease. Vitamin B1 plays an essential role in energy transformation. Thiamin diphosphate (TDP) is a coenzyme necessary for the conversion of carbohydrates to energy. Since U.S. men currently consume about 45% of their total calories from carbohydrates, vitamin B1 optimization in the diet is desirable.

Along with vitamin B6, vitamin B12 and folic acid supplementation help modulate blood levels of homocysteine and as such will be useful components in the dietary supplement formulations of the present invention. Vitamin D (calciferol) is essential for formation of the skeleton and for mineral homeostasis. Without vitamin D, the small intestine cannot absorb adequate calcium regardless of how much calcium is available for absorption. Thus, vitamin D is indicated as a component of a nutritional supplement to help build strong bones and teeth.

Further, to increase the palatability of a food product, it may be desirable to add flavors, sweetening agents, binders or bulking agents. In addition other flavorings and additives well known to those of skill in the art also may be added to the formulations to make them more palatable.

Flavors which can optionally be added to the present compositions are those well-known in the pharmaceutical art. Examples include, but are not limited to, synthetic flavor oils, and/or oils from plants leaves, flowers, fruits and so forth, and combinations thereof are useful. Examples of flavor oils include, but are not limited to, spearmint oil, peppermint oil, cinnamon oil, and oil of wintergreen (methylsalicylate). Also useful are artificial, natural or synthetic fruit flavors such as citrus oils including lemon, orange, grape, lime, and grapefruit, and fruit essences including apple, strawberry, cherry, pineapple and so forth.

Sweetening agents can be selected from a wide range of materials such as water-soluble sweetening agents, water-soluble artificial sweeteners, and dipeptide-based sweeteners, including salts thereof and mixtures thereof, without limitation.

Binders can be selected from a wide range of materials such as hydroxypropylmethylcellulose, ethylcellulose, or other suitable cellulose derivatives, povidone, acrylic and methacrylic acid co-polymers, pharmaceutical glaze, gums (e.g., gum tragacanth), milk derivatives (e.g., whey), starches (e.g., corn starch) or gelatin, and derivatives, as well as other conventional binders well-known to persons skilled in the art. Examples of bulking substances include, but are not limited to, sugar, lactose, gelatin, starch, and silicon dioxide.

When ingested in a solid form, the nutraceutical composition of the invention may additionally contain a solid carrier such as a gelatin or an adjuvant. When administered in liquid form, a liquid carrier such as water, petroleum, oils of animal or plant origin such as peanut oil, mineral oil, soybean oil, or sesame oil, or synthetic oils may be added. The nutraceutical composition of the present invention may also contain stabilizers, preservatives, buffers, antioxidants, or other additives known to those of skill in the art.

The composition of the invention may contain a variety of other beneficial components including but not limited to essential fatty acids, vitamins and minerals. These components should be well known to those of skill in the art, however, without being bound to any particularly formulations or content the present section provides a brief discussion of components that could form part of the food supplements of the present invention. Additional disclosure describing the contents and production of nutritional supplements may be found in e.g., U.S. Pat. No. 5,902,797; U.S. Pat. No. 5,834,048; U.S. Pat. No. 5,817,350; U.S. Pat. No. 5,792,461; U.S. Pat. No. 5,707,657 and U.S. Pat. No. 5,656,312 (each incorporated herein by reference

Typically, the natural food supplement of the present invention are preferably administered two times per day, preferably once in the morning and once in the evening. A typical treatment regime for the natural food supplement will continue for four to eight weeks. Depending on such factors as the medical condition being treated and the response of the patient, the treatment regime may be extended. A natural food supplement of the present invention will typically be consumed in two servings per day as either a meal replacement or as a snack between meals. A serving size for a natural food supplement of the present invention will preferably be in the range of from about 45 grams to about 60 grams and will provide from about 180 calories to about 220 calories to the consumer. In a presently preferred treatment regimen, a person or animal in need of treatment is provided with two servings of a natural food supplement of the present invention per day.

The compositions of the invention may be administered under the supervision of a medical specialist, or may be self-administered.

### EXAMPLES

### Example 1

### In Vitro Identification of Natural MMP Inhibitors

In excess of 80 botanical extracts (BE), were screened in vitro for inhibition against MMP-2, 8, & 9 proteolytic activity. Individual calorimetric assay kits (BIOMOL) were used according to the manufacturer's instructions to measure the proteolytic activity of MMP's-2, 8, and 9 in the presence or absence of individual extracts. The known MMP-inhibitor, Doxycycline was used as an internal standard for comparison of inhibiting activity.

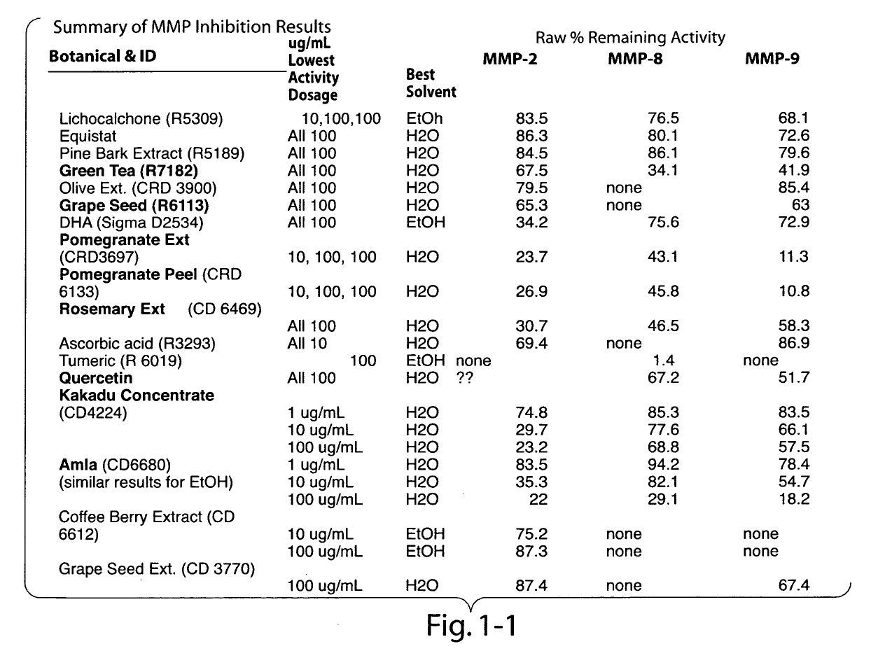

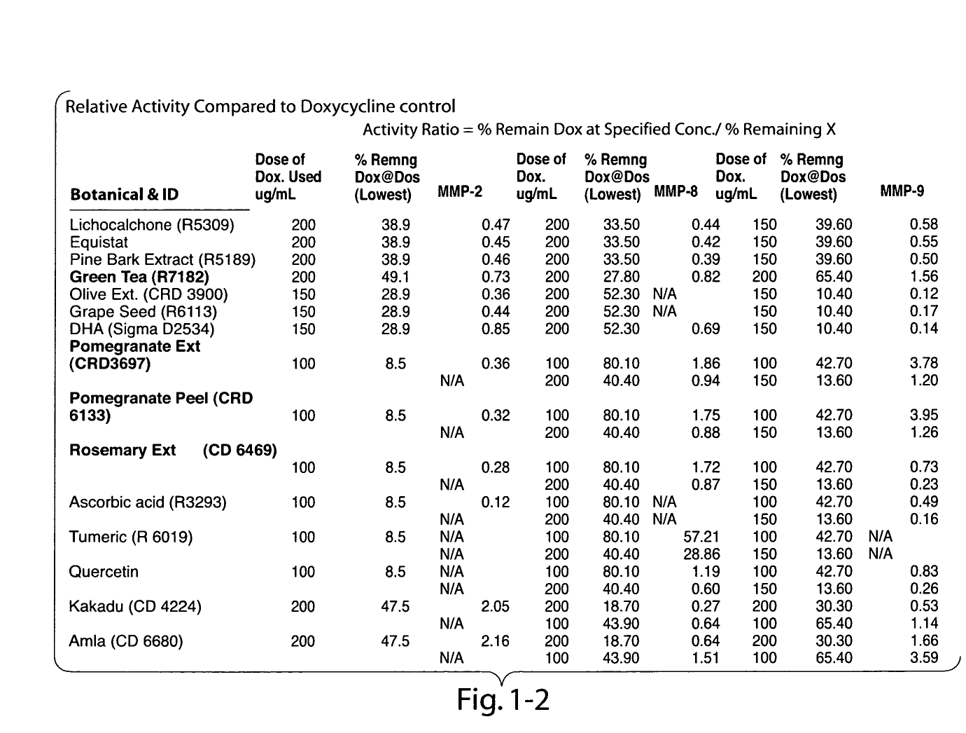

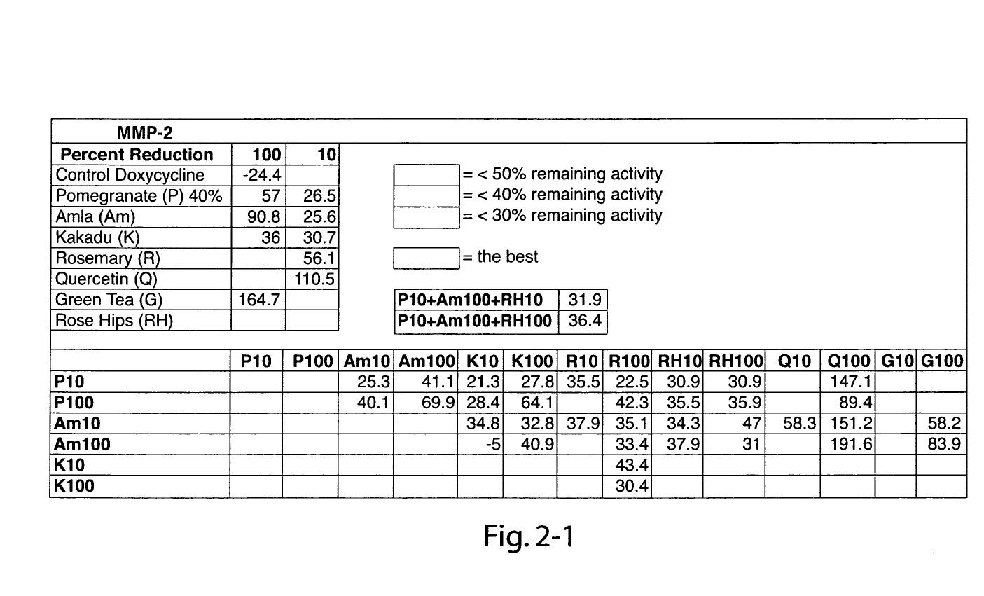

The results of these studies are shown in FIG. 1. Of the compound screened three are strong lead types and three are moderate lead types (potential secondary or blend ingredients), all of which showed inhibiting activity in excess of Doxycycline for one or more MMP's tested. (See Table 1)

TABLE 1

| | | Inhib. Relative to Doxy. | |
|---|---|---|---|
| | | (Fold = % inhib X/% | |
| | | inhib Dox | |
| | BE | MMP-2 or 9 | MMP-8 |
| Strong Leads: | A) Pomegranate ext. | 3.78 | 1.86 |
| | B) Pomegranate Peel Ext. | 3.95 | 1.75 |
| | C) Amla Ext. | 3.59 | 1.51 |
| | D) Rosemary Extract | 0.73 | 1.72 |
| Moderate Leads: | E) Quercetin | 1.56 | 0.82 |
| | F) Kakadu Extract | 0.64 | 1.14 |
| | G) Green Tea (40% ECGC) | 0.83 | 1.19 |

Six (6) of these ingredients were chosen to go back into the same assay system to show: 1) repeated activity seen in first round screening, and 2) potential interference between combinations of these ingredients in the presence of the intended ingredient known to inhibit IL-1 activity. We were able to identify combinations of ingredients which included the intended IL-1 inhibitor that showed inhibition equal (no activity interference) to or more potent than Doxycycline against MMP-8 and MMP-2 or -9.

From the studies of repeated activity and lack of interference, we were able to identify three lead ingredients for further development (A, C, D; A & B were two forms of the same dried extract). These were tested in a second laboratory, using a more sensitive in vitro (non-cellular) assay system, again for individual activity and interference/synergy among MMP-inhibitors and the IL-1-inhibitor against all three forms of MMP. These assays used enzyme kinetic dose curves to determine the IC₅₀ of each extract, Doxycyclin, and combinations of extracts (including the IL-1-inhibitor, e.g., Rosehips). Two of the lead extracts (A,C) demonstrated comparable or better proteolytic enzyme inhibiting activity compared to Doxycycline under this more rigorous assay system, and one (D) failed to confirm effective inhibiting potency. As expected, the IL-1 inhibitor did not show activity. Samples of another extract, Lichochalone LR-15 (a licorice extract) were also tested from earlier work in this lab and it confirmed activity comparable to Doxycycline against MMP-8.

IC₅₀ for MMP Inhibitors (μg/mL)

| | Enzymes | | | | | |
|---|---|---|---|---|---|---|
| | | | | | IL-1 | |
| Substance | DOX | A | C | D | Inhib. | Old |
| MMP-2 | 32 | 2.2 | 20 | No Effect | 220 | No Effect |
| MMP-9 | 40 | 7.5 | 60 | No Effect | No Effect | No Effect |
| MMP-8 | 35 | 50 | 150 | 95 | No Effect | 26 |

### Example 2

### Interference Studies

Individual calorimetric assay kits (BIOMOL) were used according to the manufacturer's instructions to measure the proteolytic activity of MMP's-2, 8, and 9 in the presence or absence of individual extracts and define mixtures of extracts. The known MMP-inhibitor, Doxycycline was used as an internal standard for comparison of inhibiting activity.

The following final concentrations of individual ingredients are combined (along with additional confirmatory test of the “neat” ingredients). Again all tests include a “gold standard” positive control (Doxycycline). The purpose of this interference testing is to see whether combined ingredients result in different levels of inhibition for all three MMP's (2, 8, 9) than found for individual ingredients. All samples are dissolved or extracted in water where practical and otherwise specified.

Series 1.

| | | |
|---|---|---|
| Pomegranate (P) @ 10 | P10 + Am10 | Amla (Am)@ 10 μg/ml |
| μg/ml | | |
| P @ 100 μg/ml | P10 + Am100 | Am @ 100 μg/ml |
| | P100 + Am100 | |
| | P100 + GAm10 | |

Series 2

| | | |
|---|---|---|
| Pomegranate (P) @ 10 | P10 + K10 | Kakadu) (K) |
| μg/ml-Done above | | @ 10 μg/ml |
| N/A done above | P10 + K100 | K @ 100 μg/ml |
| | P100 + K10 | |
| | P100 + K100 | |

Series 3

| | | |
|---|---|---|
| Pomegranate (P) @ 10 | P10 + R10 | Rosemary (R) |
| μg/ml-Done above | | @ 10 μg/ml |
| N/A done above | P10 + R100 | R @ 100 μg/ml |
| | P100 + R100 | |

Series 4

| | | |
|---|---|---|
| Amla (Am) | Am100 + K10 | Kakadu (K) |
| N/A done above | Am10 + K10 | N/A done above |
| | Am10 + K100 | |
| | Am100 + K100 | |

Series 5

| | | |
|---|---|---|
| Pomegranate (P) | P10 + Q100 | Quercetin (Q) |
| | | @ 10 μg/ml |
| N/A done above | P100 + Q100 | Q @ 100 μg/ml |

Series 6

| | | |
|---|---|---|
| Amla (Am) | Am10 + R10 | Rosemary (R) |
| @ 10 μg/ml-Done above | | @ 10 μg/ml- Done above |
| N/A done above | Am10 + R100 | N/A done above |
| | Am100 + R100 | |

Series 7

| | | |
|---|---|---|
| Amla (Am) | Am10 + Q10 | Quercetin (Q) |
| @ 10 μg/ml-Done above | | @ 10 μg/ml- Done above |
| N/A done above | Am10 + Q100 | Q @ 100 μg/ml- Done |
| | | above N/A |
| | Am100 + Q100 | |

Series 8

| | | |
|---|---|---|
| Amla (Am) | Am10 + Q100 | Green Tea (R7182) |
| @ 10 μg/ml-Done | | @ 100 μg/ml- |
| above | | |
| N/A done above | Am100 + Q100 | |

Series 9

| | | |
|---|---|---|
| Kakadu (K) | K10 + R10 | Rosemary (R) |
| | | @ 10 μg/ml- Done |
| | | above |
| N/A done above | K100 + R100 | N/A done above |

Series 10: Interference of Rosehips (R)

| | Amla (Am) + | Pomegranate + |
|---|---|---|
| Pomegranate (P) + | Rosehips (R) | Amla (Am) + |
| Rosehips (R) All in ug/ml | All in ug/ml | Rosehips (R) All in ug/ml |
| P10 + R10 | A10 + R10 | P10 + A100 + R10 |
| P10 + R100 | A10 + R100 | P10 + A100 + R100 |
| P100 + R10 | A100 + R10 | |
| P100 + R100 | A100 + R100 | |

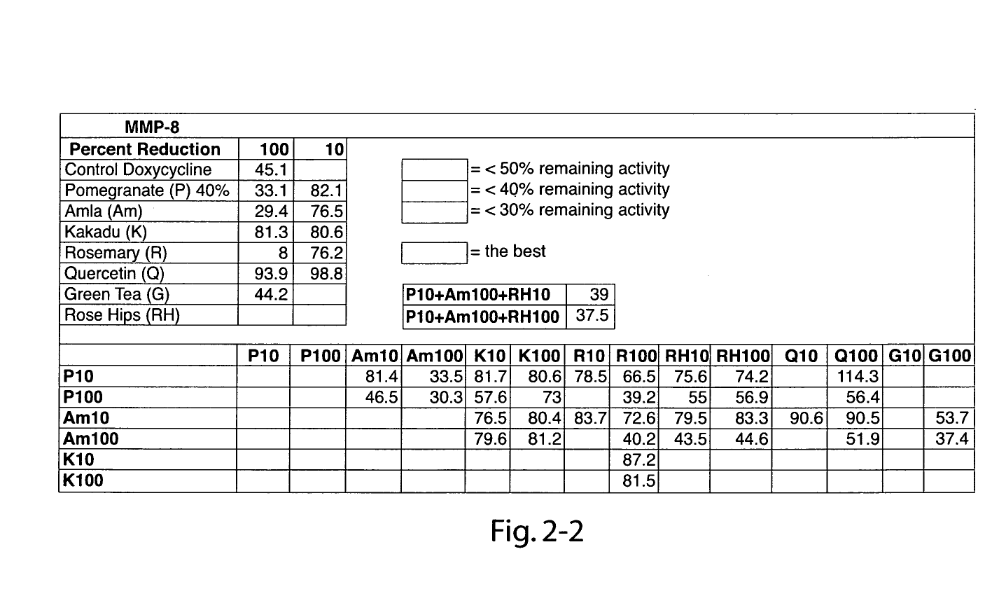

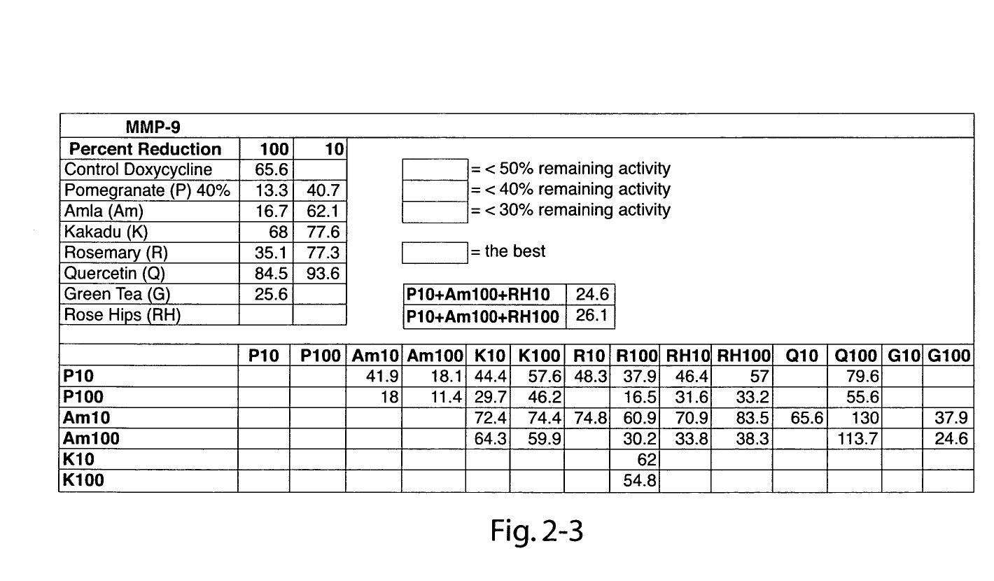

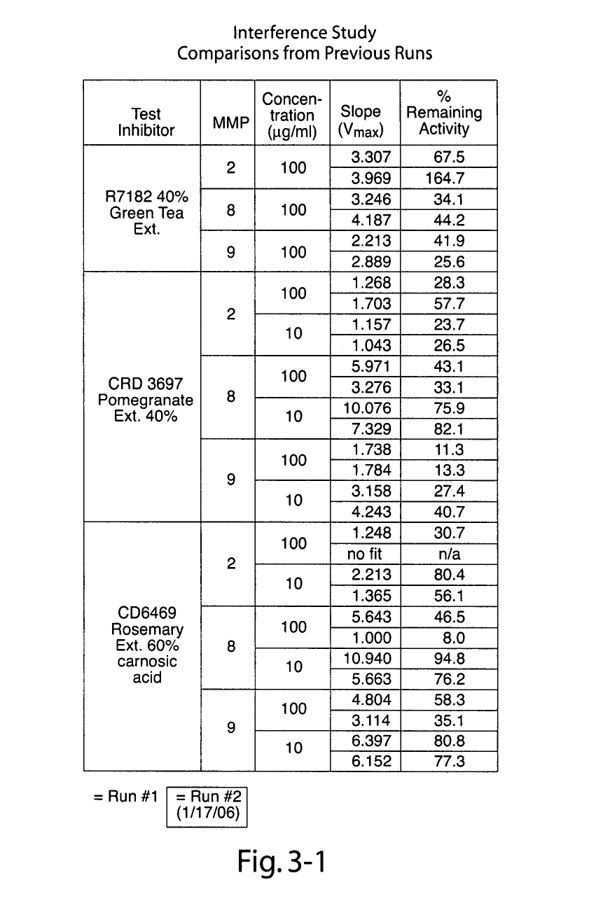

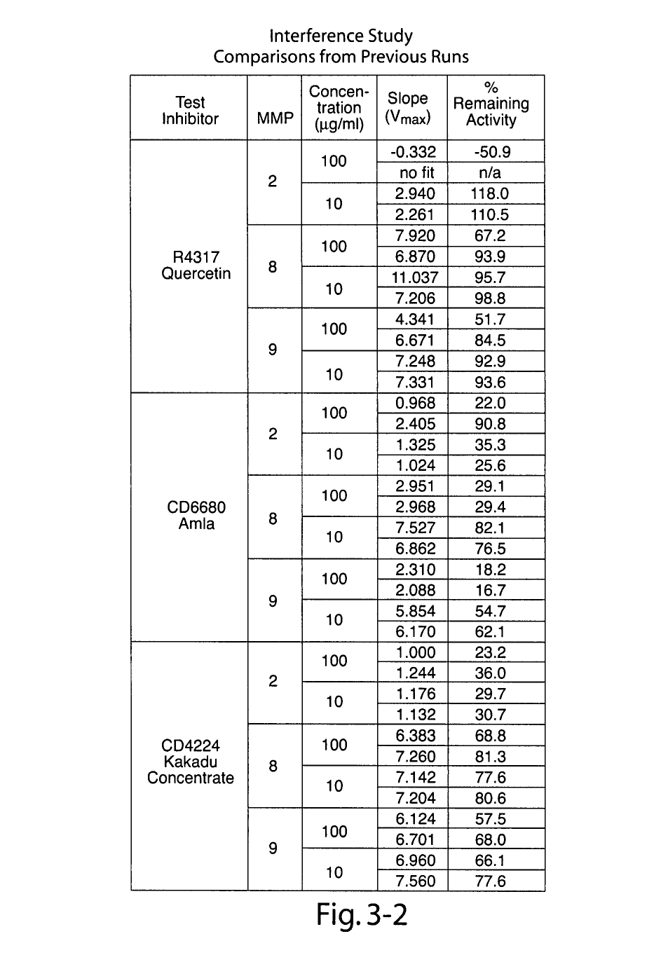

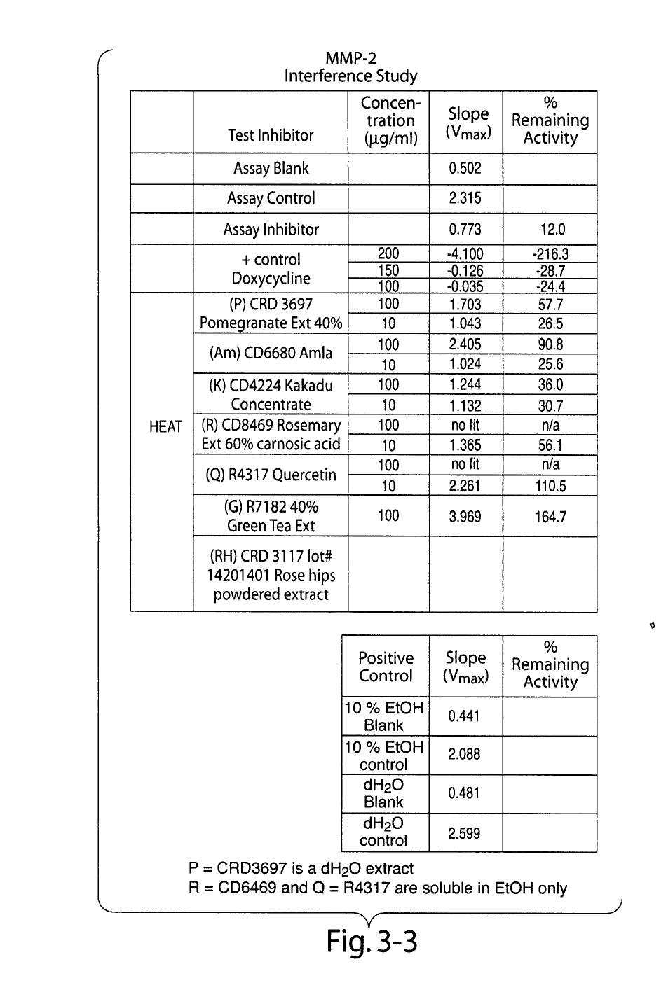

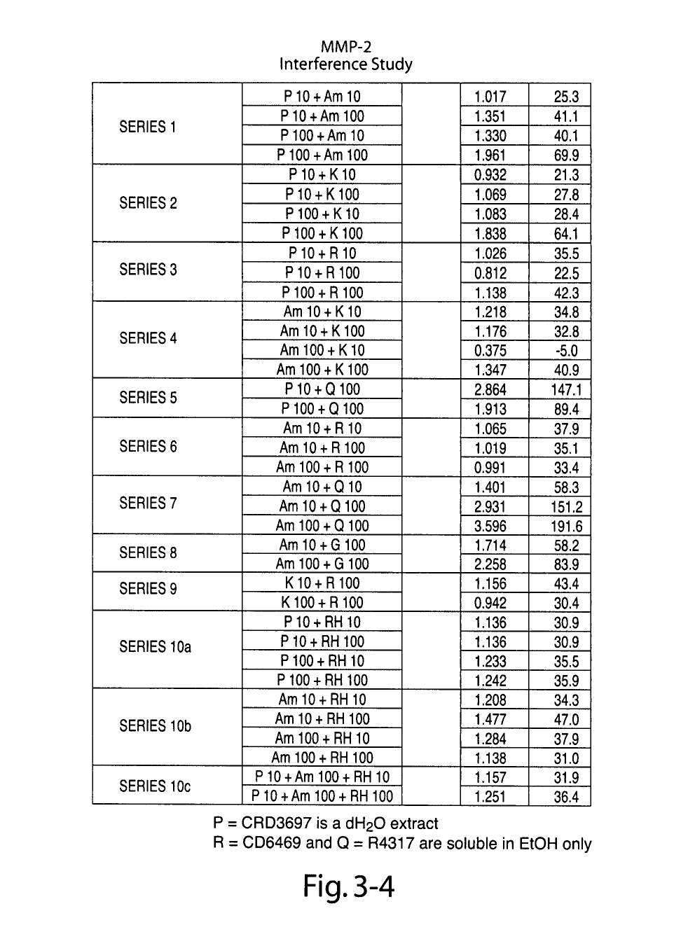

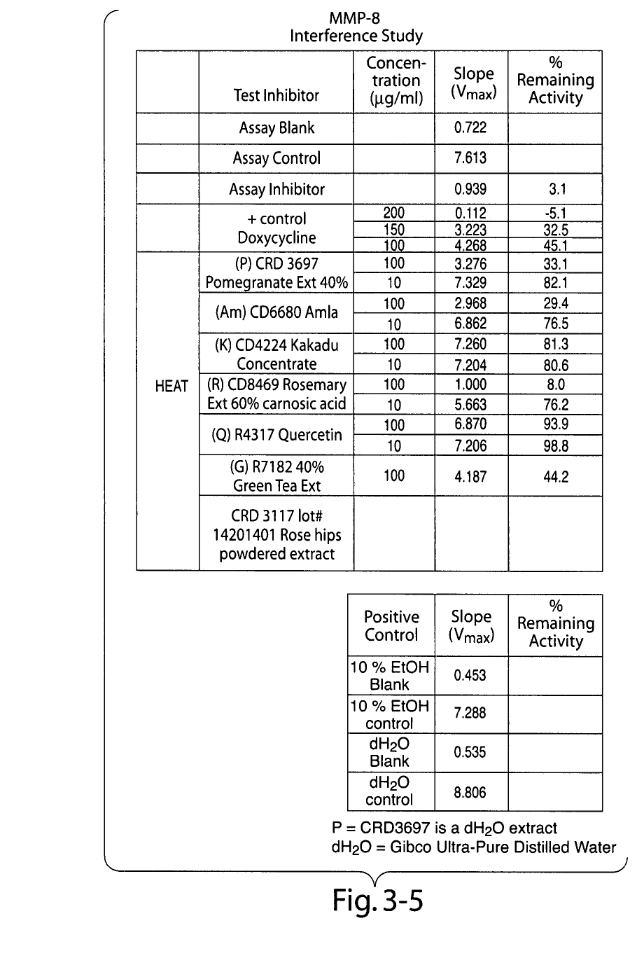

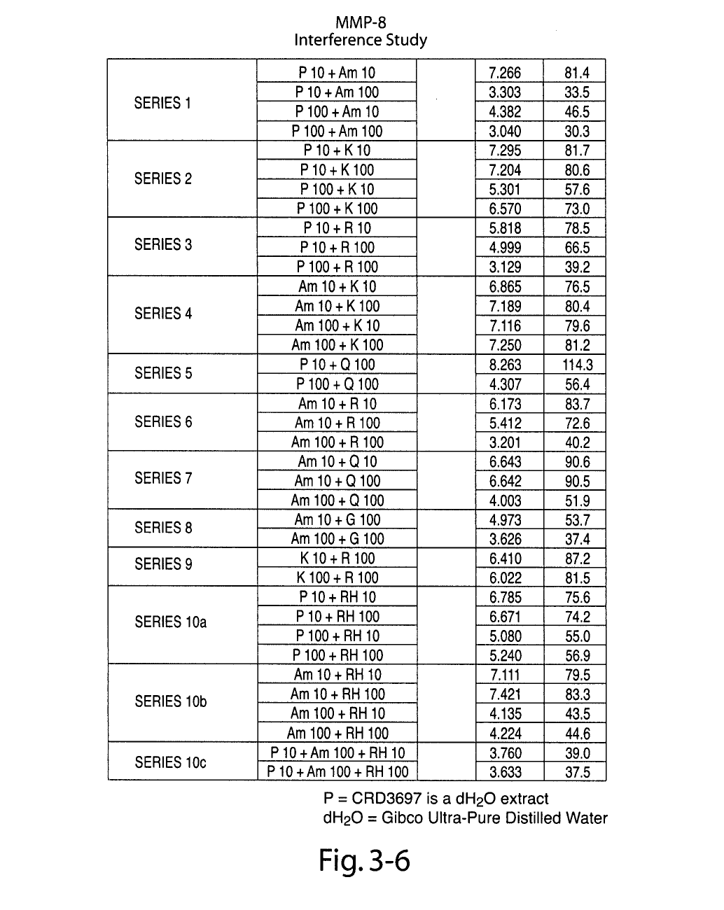

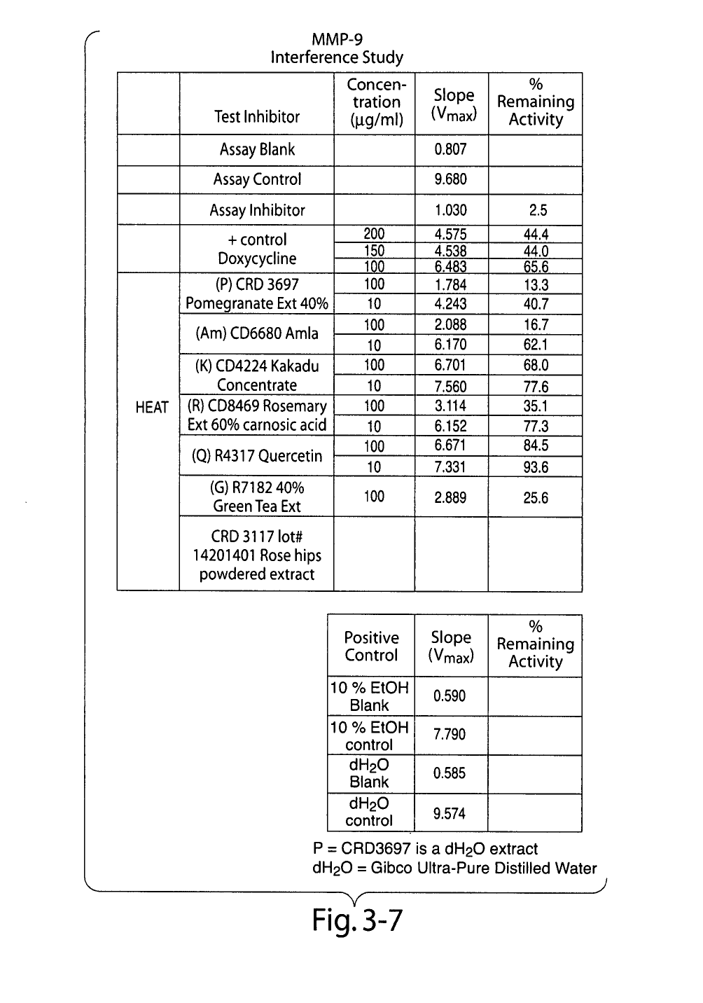

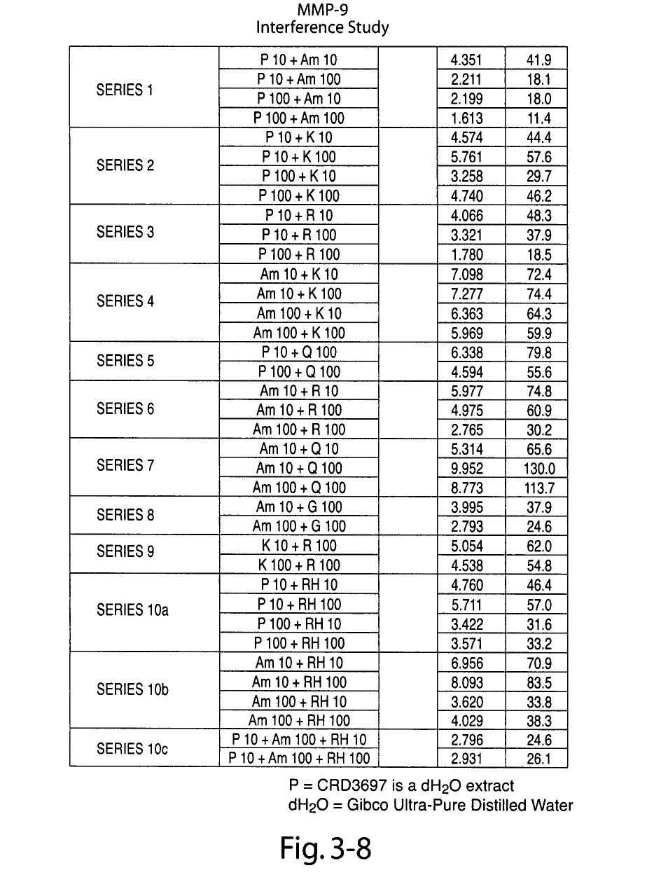

The results of the interference studies are shown in FIGS. 2 and 3.

### Example 3

### Evaluation or Rosehips for IL-1 Inhibitory Activity

The chosen ingredient for the inhibition of IL-1, Rosehips was shown to have an IC₅₀ of <1.0 ug/mL in a cell-based in vitro assay screen. This ingredient was further tested and found active in clinical trials measuring subject peripheral blood monocyte IL-1 gene activity. This was not unexpected, since prior small studies in the literature found this extract effective against pain in knee osteoarthritis, and a separate study showed it could significantly lower CRP levels. This is the ingredient, which has been tested in our in vitro assays for interference against MMP inhibition.

### Example 4

### Evaluation of Inhibitor Characteristics

To better understand the properties of the extracts as a basis for the enzymatic assays, the solubility of the potential inhibitory ingredients Pomegranate Extract (CRD 3697) (P) [water Soluble), Amla Extract (CD 6680) (Am) [Water Soluble], Rosemary Extract (CD 6469) (R) [EtOH Soluble; lipophillic, not water soluble] and Rosehips (RH) (CRD 3117) [Water Soluble] were tested in water and in ethanol (R only). Briefly, samples were added to tubes and dissolved overnight in water or ethanol on a shaker at RT ° C. or in hot water (˜90° C.) for 30 min followed by RT ° C. After centrifugation, the dissolved and undissolved fractions were separated and weighed following lyophilization. In addition to water and ethanol similar assessments defined the solubility in the MMP assay buffer (EAB). Results are presented in Table 2.

For the subsequent assays, only the soluble phase of the extracts was used to test the inhibition of MMPs. We were able to test all inhibitors over a concentration range of 0.2 up to ˜400 μg/ml.

TABLE 2
Solubility of Extracts in RTₒ_{_{c}} and Hot Water (H₂O)
and in Enzyme Assay Buffer (EAB).

| | | Results | | | | | |
|---|---|---|---|---|---|---|---|
| | | (Mean of 2 expmts.) | | Experimental data | | | |
| | | Solubility | Solubility | Concentration | Samples | Soluble | Insoluble |
| Extracts | Solution | (mg/ml) | (%) | used (mg/ml) | (mg) | (mg) | (mg) |
| P | H₂O | 11.0 | 55 | 20 | 20.3 | 11.1 | 8.9 |
| P | Hot H₂O | 5.4 | 53.6 | 10 | 11.9 | 6.2 | 5.6 |
| | | | | | 12.7 | 7.0 | 5.4 |
| P | EAB | 4.1 | 41.0 | 10 | 10.8 | 4.4 | 5.9 |
| | | | | | 12.7 | 5.2 | 7.4 |
| AM | H₂O | 16.1 | 80.4 | 20 | 24.0 | 19.3 | 4.0 |
| AM | Hot H₂O | 8.0 | 79.7 | 10 | 12.2 | 9.4 | 1.4 |
| | | | | | 11.4 | 9.4 | 1.7 |
| AM | EAB | 7.1 | 71.0 | 10 | 12.3 | 9.2 | 3.4 |
| | | | | | 11.9 | 8.1 | 3.5 |
| R | H₂O | 9.8 | 98.1 | 10 | 12.5 | 12.3 | 0.3 |
| | | | | | 13.4 | 13.2 | <0.1 |
| R | EAB | 9.8 | 98.0 | 10 | 12.5 | 12.2 | <0.1 |
| | | | | | 11.9 | 11.7 | 0.2 |
| R | EtOH | 8.2 | 41.0 | 20 | 20.6 | 8.4 | 11.8 |
| RH | H₂O | 18.8 | 94.0 | 20 | 19.7 | 18.5 | 0.4 |
| RH | EAB | 9.8 | 97.7 | 10 | 11.3 | 11.1 | 0.5 |
| | | | | | 11.1 | 10.8 | <0.1 |

### Example 5

### Evaluation of Enzymatic Inhibitory Activity

Enzymes Tested

MMP-2 and MMP-9: Recombinant MMP-2 and MMP-9 were expressed as recombinant proteins and purified in constitutively active forms.

MMP-8: Purified human neutrophil collagenase was purchased from EMD

Biosciences/Calbiochem, cat # 444229. Activation of MMP-8 was required for the experiments and was performed using aminophenylmercuric acetate (APMA) for 2 h at 37° C.

Substrates for Enzymatic Assays

DQ gelatin: A highly labeled porcine gelatin substrate (Molecular Probes, Eugine, Oreg.) was used for the enzyme assays with MMP-2 and MMP-9. The substrate degradation was measured with λex at 494 nm and λem at 515 nm

Peptide substrate: The fluorescent peptide substrate Mca-Pro-Leu-Gly-Leu-Dnp-Ala-Arg-NH2 was for measuring the MMP-8 activities (Peptides International, Louisville, Ky.). The Mca is a fluorescent group and Dnp is a quencher. When the cleavage occurs, a strong fluorescent signal is released and can be measured with λex at 328 nm and λem at 393 nm.

Enzyme Assays

To reduce any self-degradation of the gelatin substrate, all assays were performed at 22° C. The enzyme assay buffer (EAB) used for MMP-2, MMP-9, and MMP-8 was 50 mM Tris, pH 7.0, 200 mM NaCl, 5 mM CaCl₂, 1 μM ZnCl₂, 0.05% Brij 35. Assays were performed under conditions where the substrates were not rate limiting. Once optimal conditions were established, substrate and enzyme conditions were maintained constant and the reactions were repeated in the presence of concentration ranges of the individual inhibitors alone or in combination. From plots of inhibitor concentrations versus MMP activities (RFU, relative fluorescent units), the inhibitor concentrations resulting in 50% enzyme inhibition (IC₅₀) were defined.

Inhibition of MMP-2 Activities

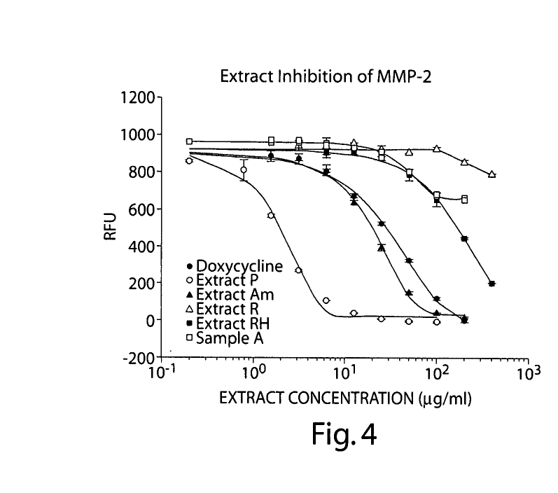

Extracts P and AM inhibited MMP-2 significantly. While AM inhibition of MMP-2 corresponded to that of doxycycline (IC₅₀ for AM 20 μg/ml compared to 32 μg/ml for Doxycycline). However, extract P provided significantly stronger inhibition of MMP-2 with an IC₅₀ of 2.2 μg/ml. I Virtually no inhibition was detected for R and A and the inhibition was weak for RH (IC₅₀ 220 μg/ml). (See Table 3, FIG. 4)

Inhibition of MMP-9 Activities

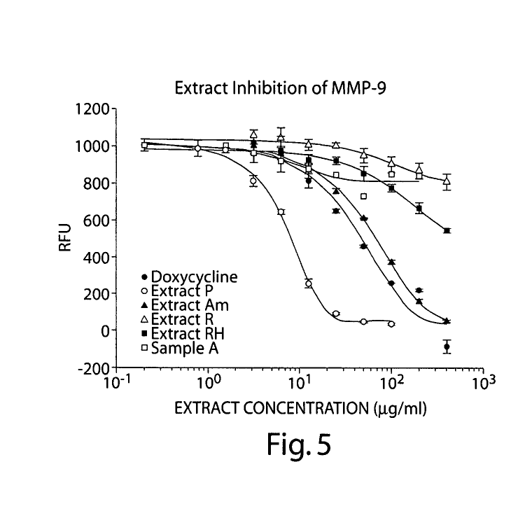

The general trend for the inhibition of MMP-9 corresponded to that observed for MMP-2. Thus, a strong inhibition occurred with P (IC₅₀ 7.5 μg/ml). There was a less pronounced effect from AM (IC₅₀ 60 μg/ml). RH, R, and A did not have any demonstrable inhibition of MMP-9. (See Table 3 and FIG. 5)

Inhibition of MMP-8 Activities

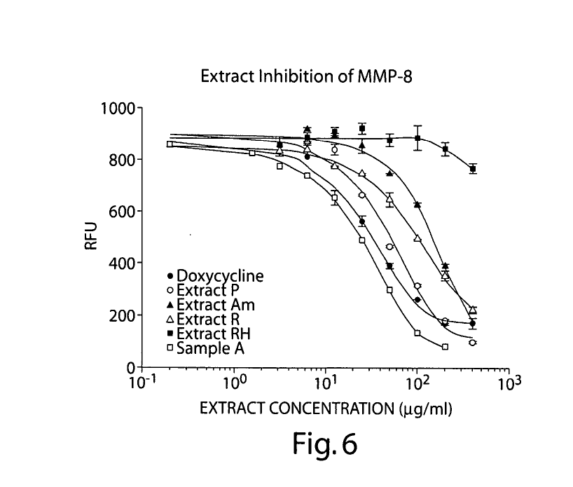

Compared to MMP-2 and MMP-9, the extracts had clearly different effects on MMP-8. Compound A (Lichochalone LR-15, a licorice extract was most efficient on MMP-8 (IC50 26 μg/ml) which was slightly better than doxycycline. Note that A had no inhibition of MMP-2 and MMP-9. However, extract P which had very strong effects on MMP-2 and MMP-9 had a lower IC₅₀ of 50 μg/ml for MMP-8. Extracts R and AM had weaker effects on MMP-8 with IC₅₀ of 95 μg/ml and 150 μg/ml, respectively. RH had no demonstrable inhibition of MMP-8. (See Table 3 and FIG. 6)

TABLE 3
IC₅₀ for 6 tested MMP Inhibitors (μg/ml)

| | Enzymes | | | | | |
|---|---|---|---|---|---|---|
| Substances | DOX | P | AM | R | RH | A (old) |
| MMP-2 | 32 μg/ml* | 2.2 μg/ml | 20 μg/ml | No effect | 220 ig/ml | No effect |
| MMP-9 | ˜40 μg/ml | 7.5 μg/ml | 60 μg/ml | No effect | No effect | No effect |
| MMP-8 | 35 μg/ml | 50 μg/ml | 150 μg/ml | 95 μg/ml | No effect | 26 μg/ml |

### Example 6

### Evaluation of the Synergistic Effect of the Inhibitors

To better understand potential interactions of the inhibitors, performed several additional experiments were performed. After the initial experiments with P alone, we proceeded to use the conditions and concentrations of P in the linear range of the assays that yielded 50% inhibition (˜IC₅₀). Concurrently, a range of concentrations of R, RH, or AM were then added to the reactions for MMP-2, MMP-9 and MMP-8. Reported are here the concentrations for the “secondary” inhibitors required to gain an additional reduction by 50% when added in conjunction with P.

Interactions for MMP-2

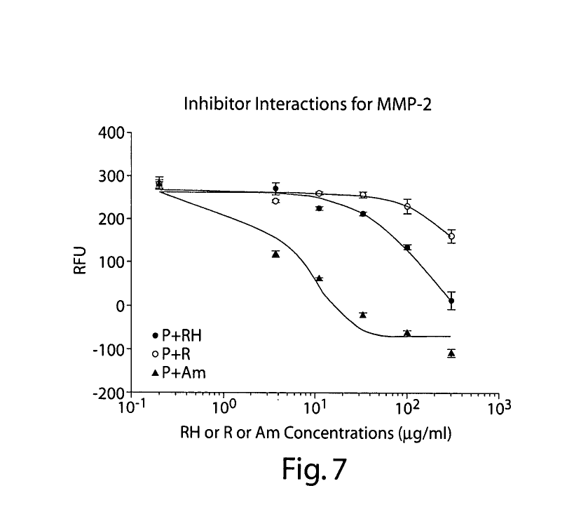

Extract R which did not inhibit MMP-2 also had virtually no effect in the combined assay. However, AM which had substantial effect on MMP-2 alone produced an additional reduction by 50% at a low concentration of 9 μg/ml. RH, a weak inhibitor, of MMP-2, induced a similar added inhibition at a concentration of 100 μg/ml. (Table 4, FIG. 7)

Interactions for MMP-9

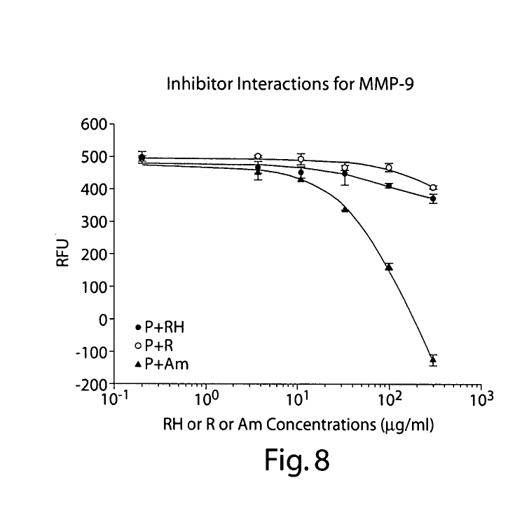

R and RH had negligible additive effect to that of P. Likewise, AM had little effect in the combined system and required 90 μg/ml to further reduce the inhibition by 50% although the IC₅₀ for single use of AM was ˜60 μg/ml. (Table 4, FIG. 8)

Interactions for MMP-8

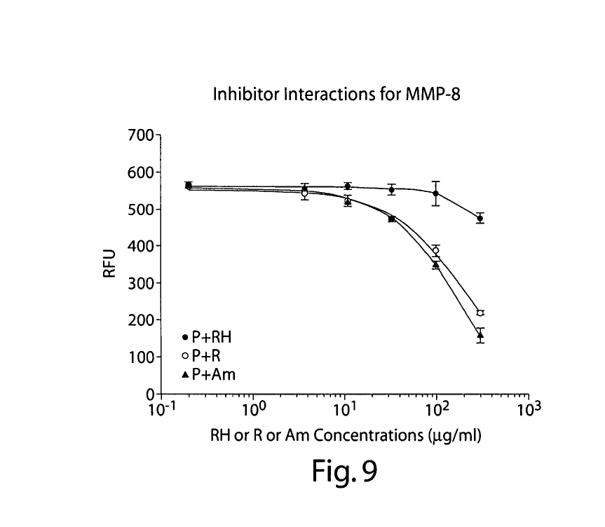

RH which had no effect alone on MMP-8 also did not augment the inhibition of P on MMP-8. Extracts R and AM also had weak additive inhibition on MMP-8 which was consistent with their weak inhibition of MMP-8 when used alone. (Table 4, FIG. 9)

Thus, adding the secondary inhibitors did augment the overall inhibition.

TABLE 4
Additive Effects of Extracts R, RH, and AM
When Added to Reactions Adjusted to IC₅₀ for P.

| | MMP-2 | MMP-9 | MMP-8 |
|---|---|---|---|
| R | No effect* | No effect | 110 μg/ml |
| RH | 100 μg/ml | No effect | No effect |
| AM | 9 μg/ml | 90 μg/ml | 95 μg/ml |

*Presented are concentrations required to obtain an additive inhibition by 50%.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A food composition comprising:
a) a natural ingredient selected from the group consisting of Pomegranate Extract, Green Tea Extract, Rosemary Extract, Quercetin, Amla Extract and Kakadu Concentrate; and
b) rose hips.

2. The composition of claim 1, wherein the natural ingredient has natural matrix metalloproteinase inhibitory activity.

3. The composition of claim 1, wherein said rose hips has interleukin-1 inhibitory activity.

4. The composition of claim 2, wherein the minimum matrix metalloproteinase inhibitory concentration of the natural ingredient in the presence of rosehips is synergistically less than the minimum matrix metalloproteinase inhibitory of the natural extract alone.

5. The composition of claim 4, wherein the minimum interleukin-1 inhibitory concentration of the rose hips in the presence of the natural ingredient is synergistically less than the minimum interleukin-1 inhibitory concentration of the rose hips alone.

6. The composition of claim 1, wherein said composition is a liquid, a lozenge, a tablet a chew, a powder or a bar.

7. The composition of claim 1, wherein the green tea extract is standardized to contain at least 40 percent polyphenols.

8. The composition of claim 7, wherein the polyphenol is (−) epigallacatchein gallate.

9. The composition of claim 1, wherein said green tea extract is present at a concentration of 2.5 weight percent to about 25 weight percent of the total dry weight of the active ingredients of the composition.

10. The composition of claim 1, wherein the pomegranate extract comprises 30 to 3000 μmol of polyphenols.

11. The composition of claim 1, wherein the rosemary extract is standardized to contain at least 58 percent carnosic acid.

12. The composition of claim 1, wherein the rosemary extract is standardized to contain at least 4 percent phenoloic diterpenes

13. The composition of claim 1, wherein the quercetin ranges from about 27 to about 55 weight percent.

14. A method of treating or alleviating a symptom of periodontal disease comprising administering to a subject in need thereof the composition of claim 1.

15. The method of claim 14, wherein said subject is a human, a cat, a dog, a monkey, or a horse.
